(19) United States
(12) Patent Application Publication
(10) Pub. No.: US 2005/0214225 A1
(43) Pub. Date: Sep. 29, 2005

(54) Pharmaceutical composition and nasal rinsing device therefor

(76) Inventors: Peter Ahnblad, Stockholm (SE); Susanne Lagerqvist Ahnblad, Stockholm (SE); Robert Halasz, Stockholm (SE)

Correspondence Address: FASTH LAW OFFICES (ROLF FASTH), 26 PINECREST PLAZA, SUITE 2, SOUTHERN PINES, NC, 28387-4301, US

(21) Appl. No.: 11/104,225

(22) Filed: Apr. 12, 2005

Related U.S. Application Data
(62) Division of application No. 10/264,096, filed on Oct. 2, 2002.
Application No. 10/264,096 is a continuation-in-part of application No. 10/069,595, filed on Jun. 4, 2002.

(51) Int. Cl. A61L 9/04; A61K 35/78

(52) U.S. Cl. 424/45; 424/747; 424/742

(57) ABSTRACT

The aromatic composition is for nasal administration and has a pharmaceutically-acceptable aqueous saline solution carrier combined with sesame oil and an essential oil such as peppermint oil and eucalyptus oil. The composition is withdrawn into a syringe and an output portion is sealingly applied to the nostril before the composition is injected therein to rinse the nostril.

### PRIOR APPLICATION

This is a continuation-in-part application of U.S. patent application Ser. No. 10/069,595, filed 4 Jun. 2002.

### FIELD OF INVENTION

The present invention relates to a pharmaceutical composition for nasal administration and a nasal rinsing device therefor.

### BACKGROUND AND SUMMARY OF INVENTION

Rinsing of the nose alleviate and reduce troubles with allergies, infections and troubles after nose surgery since the rinsing flushes away allergens (allergy-forming substance), microbes (virus, bacteria), bi-products of the body (pus, mucus) and dust and soot particles. The inflammatory swelling will be reduced and after nose surgery scabs will be loosened up, whereby the healing process will be accelerated. Rinsing of the nose with salt water is a well-known method and has been recommended by doctors for at least a hundred years.

The known nasal rinsing devices on the market are a potter container for multiple use, which is expensive and has a potter outlet, and two disposable variants which are pre-filled with salt water and water from the Atlantic, respectively, which of course become very expensive to use on regular basis and whose outlet do not function satisfactory and therefore neither their ability to fill up the nose and the fact that they can only be used with the salt water that is pre-filled in the nasal rinsing device.

A first object of the present invention is to provide an outlet portion for a nasal rinsing device that quickly, comfortably and in a well functioning way may fill the nasal cavity with liquid.

A second object of the present invention is to provide an inexpensive, simple and well functioning nasal rinsing device for multiple use.

The first object is met by means of an outlet portion for a nasal rinsing device, which is characterised in that it comprises an outlet end, a connection end and nozzle-shaped channel between these ends that show a constriction and an expanded outlet. The outlet portion has the advantage that the liquid which is pressed in through the channel will leave the channel under turbulent flow, whereby the liquid quickly fills out the nasal cavity at the same time as a thin jet is avoided that sprays directly on the mucous membrane of the nasal cavity, which feels unpleasant.

The outlet portion preferably shows a circumference increased portion, for example a droplet or balloon shaped portion, at the outlet end so that the outlet portion seals against the edges of the nostril irrespective of the size of the nostril, which has the advantage that the liquid will not leak out the wrong way and that the shape of the outlet portion will help in opening up the nostril arch, which is the most narrow portion of the nostril.

Preferably, the outlet portion is made of a flexible material that is experienced as soft and comfortable against the nose, such as silicone rubber.

The shape of the channel at the connection end of the outlet portion is preferably adapted to the connection means of the nasal rinsing device, for example conical with the larges diameter at the end so that the outlet portion fits to a syringe having a conical tip.

The second object is met by means of a nasal rinsing which is characterised in that it comprises an outlet portion of the present invention and a syringe or a compressible balloon for rinsing liquid, whereby the outlet portion is provided at the tip of the syringe or the outlet opening of the balloon, which is provided with connection means. A nasal rinsing device according to any one of the two variants is simple and inexpensive to manufacture and easy to use, especially the syringe variant where it is easy to suck in the rinsing liquid into the syringe before the nasal rinsing, and easy to keep clean, especially the syringe variant.

Of course, the outlet portion may be connected to any other type of nasal rinsing means.

The rinsing liquid may for example be salt water, oil emulsion or salt water provided with a medicine. More particularly, the aromatic composition of the present invention is for nasal administration and has a pharmaceutically-acceptable aqueous saline solution carrier combined with sesame oil and essential oil such as peppermint oil and/or eucalyptus oil. The composition is withdrawn into a syringe and an output portion is sealingly applied to the nostril before the composition is injected.

### BRIEF DESCRIPTION OF DRAWINGS

The present invention will now be described by exemplifying embodiments of the present invention together with appended drawings, in which:

FIG. 1 illustrates an embodiment of an outlet portion according to the present invention of a nasal rinsing device;

FIG. 2 illustrates a first embodiment of a nasal rinsing device of syringe type according to the present invention;

FIG. 3 illustrates a second embodiment of a nasal rinsing device of balloon type according to the present invention; and

FIG. 4 illustrates a third embodiment of a nasal rinsing device according to the present invention.

### DETAILED DESCRIPTION

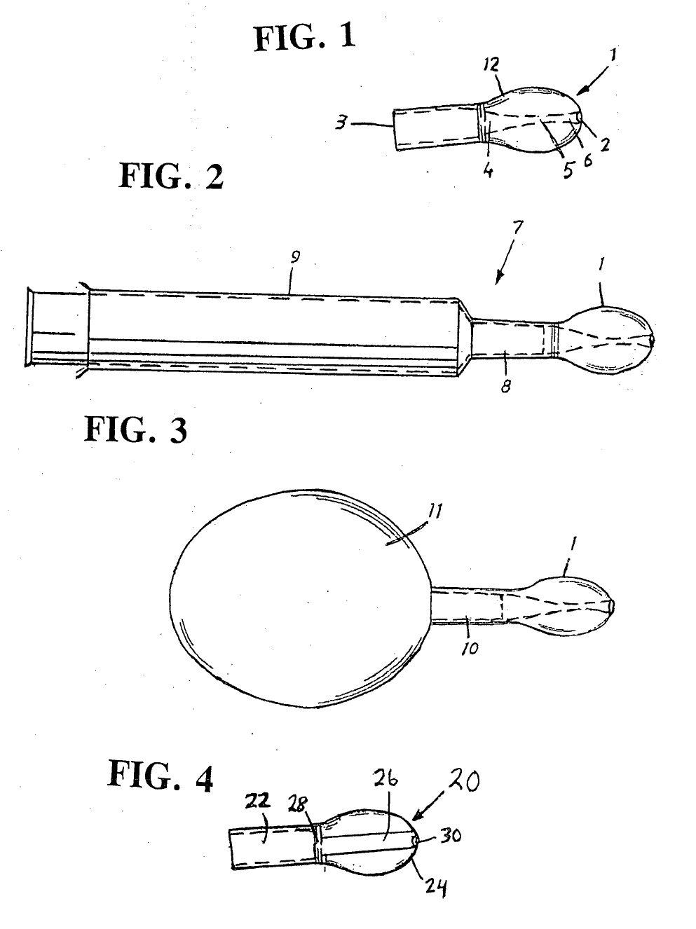

In FIG. 1 an outlet portion 1 is shown, which comprises an outlet end 2 and a connection end 3. Between these ends 2, 3 a channel 4 is arranged. The channel 4 is nozzle shaped and shows a restriction 5 in the cross section and an expanded outlet 6. At the connection end 3 the channel 4 has a shape that is adapted to the type of connection of the nasal rinsing device at which the outlet portion shall be arranged. In the shown case the channel 4 is conically shaped with the largest diameter in the connection end to fit a syringe with a conically tapering tip, for example a monoject syringe.

The outlet portion 1 has an outer shape that reminds of a lolly where the connection end 3 is the stick and the outlet end 2 is the lolly head. The outlet end 2 shows a circumference enlargement portion 12 that preferably is balloon or droplet shaped, whereby the outer diameter of the outlet portion increases from the outlet end 2 a distance inwards from this where after the outer diameter decreases. This results in the fact that the outlet portion 1 may be put into the nostril a short distance at the same time as the increasing diameter results in that the opening of the nostril fully is filled up by the outlet portion 1 so that it seals between the outlet portion 1 and the nostril. This design also results in that the outlet portion 1 suits all different sizes of nostrils. Preferably the outlet portion 1 is made of a silicone rubber.

When rinsing liquid is pressed into the outlet portion 1 from for example a syringe into the channel 4 the pressure will increase when the liquid passes the restriction 5 and will thereafter increase in velocity when the liquid reaches in the expanded outlet, whereby the flow will become turbulent.

In FIG. 2 a nasal rinsing device 7 according to a first embodiment of the present invention is shown. It comprises an outlet portion 1 provided at the tip 8 of a syringe 9, in the shown case the syringe with a conically shaped tip 8.

In FIG. 3 a nasal rinsing device according to a second embodiment of the present invention is shown. It comprises an outlet portion 1 provided at a hollow tip 10 at a balloon shaped body 11. The balloon shaped body 11, is compressible to be able to press out liquid comprised within the balloon shaped body 11 through the hollow tip 10 and out through the outlet portion.

When the outlet portion is used, a lukewarm rinsing liquid is drawn into the syringe 9 or the balloon 11 through negative pressure, whereby it is pressed out through the outlet portion 1 and further into the nostril of the user.

With reference to FIG. 4, a silicone outlet portion 20 of a third embodiment of the present invention has a hollow connection end 22 and an enlarged balloon-shaped outer end 24. The outer end 24 has a channel 26 defined therein that extends from an end section 28 of the of the connection end 22 to an outer tip 30 of the outer end 24. Preferably, the channel 24 has straight inner walls so that the channel 24 either has a constant inner diameter or a constant reduction or increase in diameter. Of course, the channel 26 may also have a variable diameter, if desired. The channel 26 extends from the tip 30 to the end section 28 so that the tip 30 is in fluid communication with the inside of the hollow connection end 22. As described above, the outer end 20 may be connected to a suitable syringe device such as the syringe 9, shown in FIG. 2, so that a suitable composition may be injected into one of the user's nostrils. Once the composition has been withdrawn into the syringe 9, the silicone portion 20 is sealed against the inside of the nostril or the bottom part of the nose and the solution containing the composition is injected with the syringe. Preferably, the portion 20 picks up some air so that the injection is turbulent and not merely linear.

Preferred pharmaceutical compositions of the present invention contain many essential components for nasal administration. The compositions contain a therapeutically effective amount of selected active aromatic agents. The preferred composition contains salt, sesame oil and essential oil such as peppermint oil or eucalyptus oil. The composition may be dissolved in water or another suitable liquid. More particularly, the pharmaceutical composition comprises a finely pulverized salt, such as sodium chloride (NaCl) or any other suitable salt, that together with a liquid, such as water, function as a pharmaceutically acceptable aqueous saline solution carrier. The saline solution carrier is physiologically compatible with the human body. The compatibility is important for the user's comfort and to prevent damage of the nasal mucosa or mucous membrane inside the nostrils. The aqueous saline solution carrier may range from 0.1% to about 99.4% weight percent. The salt is present in the solution at a level of about 0.5% to about 1.9%. More preferably, the salt amount is between 0.8% and 1.0% and most preferably about 0.9% weight percent that is very close to the salt level of the human body.

The first essential component of the present invention is sesame oil or any other suitable lubricating oil substances. The sesame oil could be cold pressed oil that contains vitamin E that is a known antioxidant. Antioxidants may be used to discard undesirable substances that are produced by the body. The antioxidants may be useful to neutralize free radical O₂ negative molecules that may exist in the body so that the oxygen becomes neutral. The sesame oil also contains a large amount of triglycerides such as lino-acid that are naturally included in the outer parts of the cells of the mucous membrane. The sesame oil may also be used to lubricate the mucous membrane and to prevent any undesirable dryness of the mucous membrane. The rinsing of the nose with just conventional salt solutions has a tendency to dry out the mucous membrane. The first sesame oil component comprises from about 0.02% to about 0.10%, preferably from about 0.03% to about 0.07% and most preferably 0.05 weight percent. If too much of the first component is added, it may be difficult to dissolve the first component in the water. If not enough of the first component is used then the antiseptic and decongestant function of the first component may be lost.

The second component of the present invention comprises an aromatic antiseptic component that has a cleaning effect and prevents excessive growth or development of harmful bacteria. The second component is a decongestant of dried mucous and reduces swelling. The component could be an essential volatile oil such as peppermint oil and/or eucalyptus oil. The second component comprises from 0.003% to about 0.040% of the composition. More particularly, if the second component is peppermint, the component comprises from about 0.005% to about 0.040%, preferably from about 0.012% to about 0.020% and most preferably about 0.016 weight percent. If the second component is eucalyptus, the component comprises from about 0.003% to about 0.020%, preferably from about 0.006% to about 0.010% and most preferably 0.008 weight percent. If too much of the second component is used, the user may experience the solution as being too strong.

The composition of the present invention may be provided in pulverized, tablet or any other suitable form. The examples described below are further embodiments that are within the scope of the present invention. The examples are given solely for the purpose of illustration and are not to be construed as limitations of the present invention, as many variations thereof are possible without departing from the spirit and scope of the invention.

### EXAMPLE 1

A nasal composition is prepared by combining the following components:

| Ingredient | W/W % |
|---|---|
| Sodium chloride | 0.90% |
| Sesame oil | 0.05% |
| Eucalyptus oil | 0.008% |
| Water | 100.00% |

A tablet or a suitable amount of a pulverized substance is added to a glass of water and allowed to dissolve in the water. A suitable amount of mixing is preferred to make sure the composition is fully dissolved in the water. The dissolved composition is injected into a nostril with the nasal rinsing device.

### EXAMPLE 2

A nasal composition is prepared by combining the following components:

| Ingredient | W/W % |
|---|---|
| Sodium chloride | 0.9% |
| Sesame oil | 0.05% |
| Peppermint oil | 0.016% |
| Water | 100.0% |

The composition is dissolved in water prior to injecting the dissolved composition into a nostril with the nasal rinsing device.

While the present invention has been described in accordance with preferred compositions and embodiments, it is to be understood that certain substitutions and alterations may be made thereto without departing from the spirit and scope of the following claims.

1-10. (canceled)

11. A method of administering an aromatic composition into a nostril, comprising;
providing an aromatic composition comprising from 0.03% to 0.07% of sesame oil, from 0.003% to 0.040% of essential oil selected from the group consisting of peppermint oil and eucalyptus oil, from 0.8% to 1.0% of salt in a pharmaceutically-acceptable aqueous saline solution carrier;
withdrawing the composition into a nasal rinsing device, the device having a nozzle shaped channel;
placing at least a portion of the device inside a nostril; and
administering the composition with the device into the nostril to rinse the nostril.

12. The method according to claim 11 wherein the method further comprises providing the nasal rinsing device with a balloon shaped enlarged circumference portion, inserting the balloon shaped portion into the nostril and sealing the portion against the nostril.

13. The method according to claim 11 wherein the method further comprises injecting the composition into the nostril.

14. The method according to claim 13 wherein the method further comprises using the sesame oil to provide lubrication of a nasal mucous membrane.

15. The method according to claim 13 wherein the method further comprises using the sesame oil to neutralize free radical oxygen molecules and triglycerides contained in the nasal mucous membrane.

16. The method according to claim 11 wherein the method further comprises using an outlet portion of the device to seal the nostril.

17. The method according to claim 16 wherein the method further comprising pressing in a rinsing liquid into the outlet portion with a syringe.

18. The method according to claim 17 wherein the method further comprises pressing the rinsing liquid out through the outlet portion into the nostril.
